(19) United States
(12) Patent Application Publication
(10) Pub. No.: US 2006/0058537 A1
(43) Pub. Date: Mar. 16, 2006

(54) Method for the produciton of trioxane

(75) Inventors: Michael Haubs, Bad Kreuznach (DE); Matthias Goring, Hofheim (DE); Michael Hoffmockel, Niedernhausen (DE); Jurgen Lingnau, Mainz (DE); Karl-Friedrich Muck, Wiesbaden (DE)

Correspondence Address: CONNOLLY BOVE LODGE & HUTZ, LLP, P O BOX 2207, WILMINGTON, DE, 19899, US

(73) Assignee: Ticona Gmbh, kelsterbach (DE)

(21) Appl. No.: 10/538,787

(22) PCT Filed: Dec. 12, 2003
(86) PCT No.: PCT/EP03/14122

§ 371 Date: Sep. 19, 2005

(30) Foreign Application Priority Data
Dec. 13, 2002 (DE) 102 58 663.2

(51) Int. Cl. C07D 323/06

(52) U.S. Cl. 549/368

(57) ABSTRACT

The invention relates to a process for preparing trioxane from aqueous formaldehyde solutions in the presence of acidic catalysts. The reaction of the formaldehyde to give trioxane takes place in a reaction column equipped with a circulation evaporator, and the formaldehyde solution used as the starting solution is mixed with a sidestream from the bottom of the reaction column and fed to the upper section of the reaction column via a tubular reactor. The process is notable for a low energy requirement and little by-product formation.

The invention relates to a process for preparing 1,3,5-trioxane (referred to hereinbelow as trioxane) from aqueous formaldehyde solutions in the presence of acid as a catalyst with high selectivity and low energy demands.

The preparation of trioxane from aqueous formaldehyde solutions has been known for some time (cf., for example, U.S. Pat. No. 3,483,214). According to the prior art, trioxane is formed from concentrated aqueous formaldehyde solutions in the presence of acidic catalysts. Distillation of the reaction mixture provides a trioxane-rich gas phase. Further known separation processes make it possible to prepare pure trioxane therefrom.

DE-A-1,543,390 describes a process in which trioxane-containing vapor exiting from the reactor is conducted in countercurrent to a substantially fully reacted reaction mixture in a column. The reaction mixture from the reactor is particularly advantageously conducted in countercurrent to the trioxane-containing vapor. The starting material fed to the reactor is formaldehyde as a concentrated aqueous solution.

DE-A-4,035,495 discloses a process for preparing trioxane in which acetal polymers are degraded in the presence of acidic catalysts. The process is carried out in a known circulation reactor with evaporator.

However, the processes mentioned are in need of improvement in the following points:

1. Formation of by-products in the reactor

2. Energy demands in the distillation of the reaction mixture

3. Corrosion in the reactor and in the column as a result of the catalyst

It is an object of the invention to improve the process for preparing trioxane with regard to the points 1-3 mentioned.

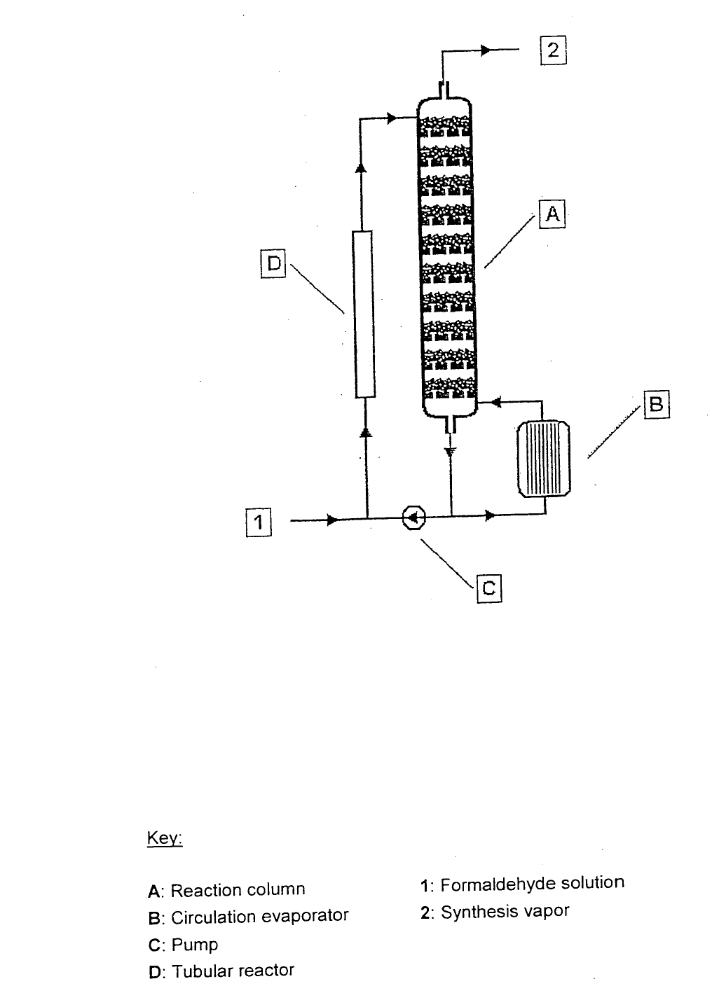

The achievement of the object is illustrated with reference to FIG. 1: The reaction mixture composed of aqueous formaldehyde solution and catalyst is disposed in the reaction column A and occupies its hold-up volume. From the bottom stream of the column A, by means of the circulation evaporator B, a vapor mixture is generated and is used to charge the reaction column A from below. A portion of the bottom stream is mixed with fresh formaldehyde solution 1 and fed to the upper section of the reaction column A as a recycle stream via the tubular reactor D with the aid of the pump C. The trioxane-containing synthesis vapor 2 is drawn off in gaseous form as the top product of the column.

As is evident from FIG. 1 and described in detail in the further text, a mixture which consists substantially of fresh, concentrated formaldehyde solution is fed to the tubular reactor. This allows a high trioxane concentration and a high space-time yield to be achieved at low catalyst concentration. The use of a low catalyst concentration reduces the corrosive action of the catalyst; the high trioxane concentration increases the trioxane concentration in the synthesis vapor and thus reduces the energy consumption, and the high space-time yield at low catalyst concentration finally suppresses the formation of by-products.

Suitable reaction columns are all known constructions. However, they have to be manufactured from a material which withstands the acidic reaction conditions. Suitable materials are, for example, nickel-based alloys, tantalum or zirconium. Plastic-coated columns are also suitable in principle.

The reaction column is further characterized by its hold-up volume Vₖ. Preference is given to constructions having a particularly high hold-up volume, since the volume Vₖ constitutes a substantial portion of the total reaction volume. Suitable columns therefore have double-cap trays or have sieve trays having high weirs.

Suitable tubular reactors are vessels, simple tubes or tube bundles. In order to achieve a uniform residence time spectrum, static mixers or coiled structures which act as mixers may be installed in the tubular reactor.

It has been found that the plant shown in FIG. 1 can be operated particularly advantageously when the following parameters are observed: when V_{FF} is the volume of fresh formaldehyde solution fed to the tubular reactor per unit time and V_{SV} is the column bottom volume fed to the tubular reactor per unit time, the V_{FF}/V_{SV} ratio is, according to the invention, between 0.5 and 20, preferably between 1 and 10 and more preferably between 2 and 5.

Together with the bottom of the column, the circulation evaporator contains a particular volume, the column bottom volume. It has been found that the process according to the invention affords particularly good results when the volume V_{US} is smaller than the hold-up volume of the reaction column. When V_{K} is the reaction volume in the column, the V_{K}/V_{US} ratio is, according to the invention, between 1 and 10 and preferably between 2 and 5.

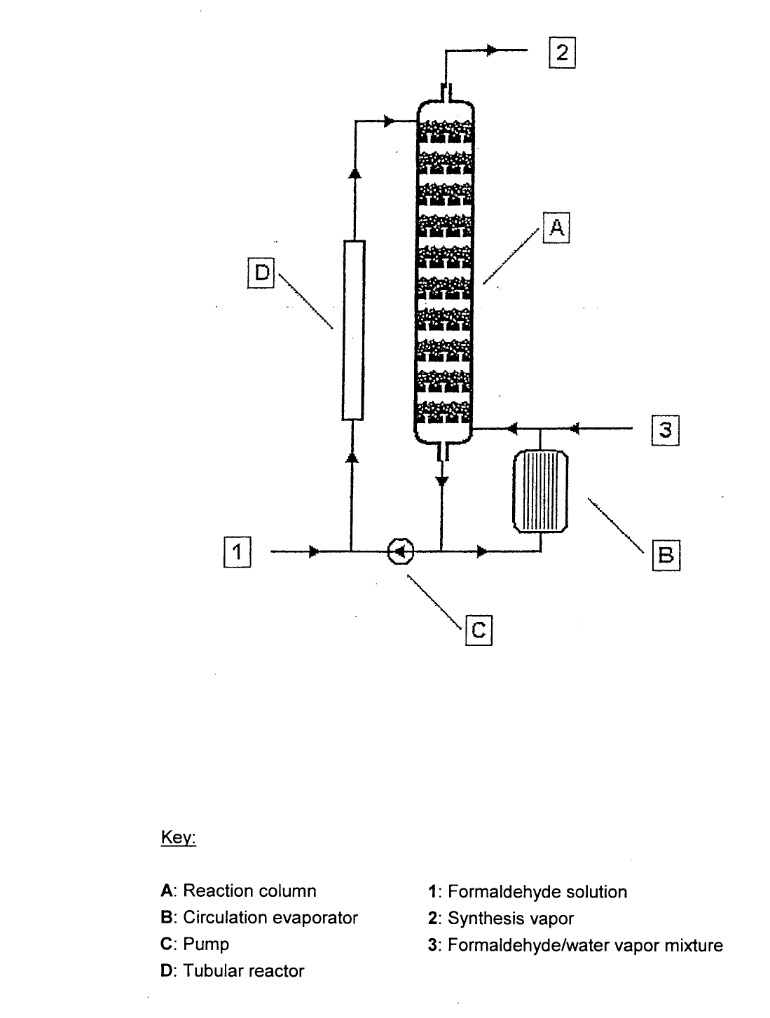

The amount of vapor generated in the circulation evaporator depends upon the inlet stream of aqueous formaldehyde solution. Experience has shown that from about 0.7 to 0.9 kg of vapor needs to be generated per kg of incoming formaldehyde solution. Some or all of the formaldehyde-containing vapor with which the lower section of the column is charged may also stem from separate sources, for example from other plant parts. In this case, the amount of vapor generated in the circulation evaporator may be reduced accordingly. Such separate vaporous formaldehyde sources are frequently available in the workup section of an industrial trioxane plant. This direct utilization of formaldehyde vapor dispenses with expensive condensers, increases the overall performance of the plant and reduces the process complexity. This advantageous embodiment of the invention is shown schematically in FIG. 2. The concentration of formaldehyde in the mixture 3 is between 35% by weight and 100% by weight, preferably between 45% by weight and 75% by weight and more preferably between 55% by weight and 65% by weight.

The reaction mixture which leaves the tubular reactor should substantially have reacted to completion, i.e. the trioxane concentration should virtually have achieved the equilibrium value. The volume of the tubular reactor is such that the average residence time in the tubular reactor is sufficient to establish equilibrium. The average residence time in the tubular reactor which is required for this purpose depends substantially upon the temperature and the catalyst concentration, and to a somewhat smaller extent also upon the formaldehyde concentration. Experience has shown that the average residence time is between 1 min and 20 min, preferably between 2 min and 10 min. The temperature at the outlet of the tubular reactor should correspond approximately to the temperature of the uppermost tray of the reaction column.

From the bottom of the reaction column, a small sidestream is appropriately removed continuously or batchwise in order to remove involatile impurities of the inlet stream and involatile by-products from the system.

Experience has shown that it is sufficient when this sidestream is about 0.1% to 1% of the inlet stream.

The concentration of the formaldehyde solution used as the starting solution is between 50% by weight and 85% by weight, preferably between 60% by weight and 80% by weight. The temperature of the solution is between 60° C. and 150° C., preferably between 70 and 130° C. At temperatures above 100° C., the solution has to be kept under elevated pressure in order to prevent formation of vapor bubbles.

Suitable catalysts are strong acids which are present dissolved or undissolved in the reaction mixture. Examples are sulfuric acid, trifluoro-methanesulfonic acid, toluenesulfonic acid or strongly acidic ion exchangers. It is also possible to use acidic zeolites or heteropolyacids. The concentration of catalyst is typically between 0.2 and 10% by weight, preferably between 0.4% by weight and 1.9% by weight. Among the soluble catalysts, preference is given to sulfuric acid.

Preferred undissolved catalysts are commercial, strongly acidic ion exchangers. They may be present suspended in the reaction mixture or be used in the form of packings.

The concentration of catalyst depends upon the concentration of the formaldehyde solutions used. At low formaldehyde concentrations, higher concentrations of catalyst are used and vice versa. When sulfuric acid is used as the catalyst, the sulfuric acid concentration is 5-10% by weight at a formaldehyde concentration of 60%, whereas the sulfuric acid concentration is from 0.2 to 4% by weight at a formaldehyde concentration of 80%.

The (average) temperature in the reaction column likewise depends upon the concentration of the formaldehyde solution used. With increasing formaldehyde concentration, the column temperature also rises. It is typically between 95° C. and 140° C., preferably between 100° C. and 125° C. At temperatures above 100° C., the column is operated under elevated pressure. The elevated pressure depends upon the formaldehyde concentration and the temperature. It is typically in the range from about 0.1 bar to 4 bar.

The synthesis vapor which leaves the reaction column comprises, in addition to trioxane, also formaldehyde, water and volatile by-products. A crucial factor for the specific energy consumption is the trioxane concentration in the synthesis vapor. When 80% by weight formaldehyde solutions are used, it is possible to attain trioxane concentrations of up to 28% by weight.

### EXAMPLES

The examples which follow are intended to illustrate the invention without restricting it.

### Example 1

Preparation of trioxane in the tubular reactor:

A 79% aqueous formaldehyde solution is mixed with concentrated sulfuric acid in a mass ratio of 100:1 and introduced into a tubular reactor at a temperature of 120° C. In the middle and at the end, the tubular reactor has sampling devices. The average residence time was calculated from the volume of the tubular reactor and the throughput by the following formula:
t=V_{RR}/V_{P}
in which:

t: average residence time in minutes

V_{RR}: volume of the tubular reactor from the start to the sampling point in liters

V_{P}: volume flow rate through the reactor in liters/min.

The samples taken were neutralized immediately with dilute alkali in order to rule out a subsequent change in the trioxane concentration. The following dependence of the trioxane concentration on the residence time in the tubular reactor was measured:

| | | |
|---|---|---|
| Residence time in minutes: | 2 | 4 |
| Trioxane concentration in % by weight: | 3.2 | 5.4 |

At a density of the reaction solution of 1.2 kg/liter, a space-time yield of trioxane in the tubular reactor of, respectively, 770 kg/m³/h and 650 kg/m³/h was calculated therefrom.

### Example 2

The experiment of example 1 was repeated except that the concentration of sulfuric acid was only 0.5% by weight. The following values were measured:

| | | |
|---|---|---|
| Residence time in minutes: | 3 | 6 |
| Trioxane concentration in % by weight: | 3.1 | 5.2 |

At a density of the reaction solution of 1.2 kg/liter, a space-time yield of trioxane in the tubular reactor of, respectively, 670 kg/m³/h and 540 kg/m³/h was calculated therefrom.

What is claimed is:

1. A process for preparing trioxane from aqueous formaldehyde solutions in the presence of acidic catalysts in a reaction column equipped with a circulation evaporator, which comprises mixing the formaldehyde solution used as a starting solution with a sidestream from the bottom of the reaction column and feeding it to the upper section of the reaction column via a tubular reactor.

2. The process as claimed in claim 1, wherein the ratio of the volume per unit time of fresh formaldehyde solution fed to the tubular reactor and the volume per unit time from the bottom of the reaction column fed to the tubular reactor is between 0.5 and 20, preferably between 1 and 10 and more preferably between 2 and 5.

3. The process as claimed in claim 1, wherein the volume of the reaction column is greater than the column bottom volume.

4. The process as claimed in claim 1, wherein the average residence time in the tubular reactor is between 1 min and 20 min, preferably between 2 min and 10 min.

5. The process as claimed in claim 1, wherein the concentration of acidic catalyst is below 2% by weight.

6. The process claim 1, wherein the pressure in the reaction column is above atmospheric pressure and preferably from 1.5 to 2.5 bar_{abs}.

7. The process as claimed in claim 1, wherein some or all of the formaldehyde vapor with which the lower section of the column is charged stems from separate sources.

8. The process as claimed in claim 1, wherein the catalyst used is a strongly acidic ion exchanger.

9. An apparatus for carrying out the process as claimed in claim 1, comprising
i) a reaction column (A),
ii) a bottom stream draw line which opens into
iii) a circulation evaporator (B) and feeds to it a portion of the bottom stream, and
iv) a draw line for the vapor mixture generated leads from the circulation evaporator into the lower section of the reaction column,
v) a draw line for the branching-off of a portion of the bottom stream leads from draw line ii) before it opens into the circulation evaporator (B) and,
vi) together with a feed line for fresh formaldehyde solution, opens into
vii) a tubular reactor (D) which opens via a draw line
viii) into the upper section of the reaction column (A).

10. The apparatus as claimed in claim 9, wherein a pump (C) is provided, with which a portion of the bottom stream and of the fresh formaldehyde solution is fed via the tubular reactor (D) to the upper section of the reaction column (A).

11. The apparatus as claimed in claim 9, wherein at least one feed line is provided, with which the formaldehyde-containing vapor which does not stem from the circulation evaporator (B) can be fed to the lower section of the reaction column (A).
